(19) United States
(12) Patent Application Publication
(10) Pub. No.: US 2026/0076725 A1
(43) Pub. Date: Mar. 19, 2026

(54) Systems and Methods for Orthopedic Fixation

(71) Applicant: IlluminOss Medical, Inc., East Providence, RI (US)

(72) Inventors: Robert A. Rabiner, Dortmund (DE); Anthony W. O'Leary, Walpole, MA (US)

(73) Assignee: IlluminOss Medical, Inc., East Providence, RI (US)

(21) Appl. No.: 19/331,324

(22) Filed: Sep. 17, 2025

Related U.S. Application Data
(60) Provisional application No. 63/695,664, filed on Sep. 17, 2024.

(51) Int. Cl. A61B 17/84; A61B 17/80; A61B 17/86; A61B 17/90

(52) U.S. Cl. CPC A61B 17/844; A61B 17/8052; A61B 17/8685; A61B 17/90

(57) ABSTRACT

Systems and methods for bone fixation are disclosed herein. According to aspects illustrated herein, there is provided a fastener for use in securing a bone plate that includes an outer member having a first outer section and a second outer section, wherein formed in the first outer section is a first bore with a first shape and formed in the second outer section is a second bore with a second shape; and an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion.

### RELATED APPLICATIONS

This application claims the benefit of and priority to U.S. Provisional Ser. No. 63/695,664, filed on Sep. 17, 2024, the entirety of this application is hereby incorporated herein by reference.

### FIELD

The embodiments disclosed herein relate to medical devices, and more particularly to monocortical orthopedic locking systems for medical applications.

### BACKGROUND

Bones form the skeleton of the body and allow the body to be supported against gravity and to move and function in the world. Bone fracture repair is the process of rejoining and realigning the ends of broken bones. Currently there are several internal approaches to repair, strengthen and support a fractured bone.

### SUMMARY

Bone fixation systems, and methods for using the systems for stabilizing portions of a weakened or fractured bone once they have been brought into corrective alignment using a photodynamic bone stabilization system (PBSS) are disclosed herein. According to aspects illustrated herein, there is provided a fastener for use in securing a bone plate that includes an outer member having a first outer section and a second outer section, wherein formed in the first outer section is a first bore with a first shape and formed in the second outer section is a second bore with a second shape; and an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion.

According to aspects illustrated herein, there is provided a bone fixation system that includes an elongated plate having an upper surface, a lower surface for positioning to face a bone, and a plate bore hole extending from the upper surface to the lower surface; and a first fastener sized to be inserted through the plate bore hole and having an outer member having a first outer section and a second outer section, wherein formed in the first outer section is a first bore with a first shape and formed in the second outer section is a second bore with a second shape; and an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion.

According to aspects illustrated herein, there is provided a method for treating a bone that includes inserting an inflatable balloon catheter into a medullary canal of the bone; expanding the balloon catheter with a biocompatible monomer; activating a visible light source to cure the monomer and form a cured internal bone fixation implant; placing a bone plate alongside the bone, the bone plate having an upper surface, a lower surface for positioning to face the bone, and a plate first hole extending from the upper surface to the lower surface; inserting a fastener through the first hole, into the underlying bone, and at least into the implant, wherein the fastener comprises: an outer member having a first outer section and a second outer section, wherein formed in the first outer section is a first bore with a first shape and formed in the second outer section is a second bore with a second shape; and an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion; engaging an effector tool with the peripheral engagement portion of the internal member of the fastener; applying a force to the effector tool to longitudinally move the internal member with respect to the outer member to cause the second outer section of the outer member to expand, wherein expansion of the second outer section of the outer member of the fastener results in fixation of the fastener to the implant.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 shows a cross-sectional view of an embodiment of components of a fastener for use with an embodiment of a monocortical orthopedic locking system of the present disclosure;

FIG. 2 shows a close-up perspective view of an outer member component of the fastener of FIG. 1;

FIGS. 3A and 3B show close-up side views showing portions of an outer member component and an internal member component of the fastener of FIG. 1. FIG. 3A shows a close-up side view showing the internal member positioned within the outer member such that the distal end of the internal member protrudes outside the distal end of the outer member in a first unexpanded state. FIG. 3B shows a close-up partial side cross-sectional view showing that the internal member, while still positioned within the outer member, has been longitudinally pulled and withdrawn into the outer member;

FIGS. 4A-4C are schematic illustrations of cross-sectional perspective views showing placement of components of a monocortical orthopedic locking system of the present disclosure during use. The monocortical orthopedic locking system (bone fixation system) comprises an elongated bone plate with bore holes that are sufficiently designed to receive a plurality of the fasteners of FIG. 1 to help stabilize a weakened or fractured bone that has been repaired with a photodynamic bone stabilization system (PBSS) resulting in a cured internal bone fixation implant. FIGS. 4A-4C show an alignment guide instrument and an effector tool which help position and fixate a fastener;

FIG. 5 is a schematic illustration of a close-up cross-sectional side view of the fastener of FIG. 1 positioned within the fractured bone and the cured internal bone fixation implant;

FIG. 6 is a schematic illustration of a close-up cross-sectional side view of the fastener of FIG. 1 positioned within the bore hole of the bone plate, the bone, and the cured internal bone fixation implant. The outer member of the fastener has been moved to an expanded position after the internal member is longitudinally moved with respect to the outer member by a pulling force, causing the distal end of the outer member to flare outward;

FIG. 7 shows a cross-section view of an embodiment of an outer member component of a fastener for use with an embodiment of a monocortical orthopedic locking system of the present disclosure;

FIG. 8 shows a cross-section view of an embodiment of an internal member component of a fastener for use with an embodiment of a monocortical orthopedic locking system of the present disclosure;

FIGS. 9A-9C show an exemplary embodiment of an alignment guide instrument and an effector tool that can be used during a method for treating a bone of the present disclosure. FIG. 9A shows a perspective view of components of a fastener of the present disclosure, such as those of FIGS. 7 and 8, along with an alignment guide instrument and an effector tool. FIG. 9B shows a perspective cross-sectional view of FIG. 9A. FIG. 9C shows a close-up perspective cross-sectional view of FIG. 9B;

FIGS. 10A and 10B show cross-sectional perspective views of an exemplary embodiment of the use of the fastener of FIG. 9B as a component of a monocortical orthopedic locking system to fixate a weakened or fractured bone having a cured internal bone fixation implant;

FIG. 11 is a schematic illustration of a close-up cross-sectional perspective view of the fastener of FIG. 10B within the bore hole of the bone plate, the bone, and the cured internal bone fixation implant. The outer member of the fastener has been moved to an expanded position after the internal member is longitudinally moved with respect to the outer member by a pushing force, causing the distal end of the outer member to flare outward;

FIGS. 12 and 13 are schematic illustrations showing a plurality of the fasteners of FIG. 10B as part of a monocortical orthopedic locking system of the present disclosure. The fasteners are positioned through the bone plate bore holes, through cortical bone and within a cured internal bone fixation implant to help stabilize the bone along a length thereof. FIG. 12 is a schematic illustration showing a cross-sectional perspective view and FIG. 13 is a schematic illustration showing a perspective view;

FIG. 14 shows an exemplary embodiment of an outer member component of a fastener for use with an embodiment of a monocortical orthopedic locking system of the present disclosure;

FIG. 15 shows a cross-sectional view of the outer member of FIG. 14;

FIGS. 16A and 16B show various view of the outer member of FIG. 14 showing the internal design;

FIG. 17 is a schematic illustration of portions of the outer member of FIG. 14;

FIG. 18 is a schematic illustration of portions of the outer member of FIG. 14;

FIG. 19 is a schematic illustration of portions of the outer member of FIG. 14;

FIGS. 20A and 20B are schematic illustrations showing the physical relationship between a first internal member, a second internal member, and an effector tool within the outer member of FIG. 14. FIG. 20A is a schematic front view and FIG. 20B is a schematic side view and in both views the schematic representation of the first digital wedge, the second distal wedge, the first proximal wedge and the second proximal wedge of the outer member have been removed for clarity;

FIGS. 21A and 21B are schematic illustrations showing the physical relationship between a first internal member and a second internal member of the outer member of FIG. 14. FIG. 21A is a schematic front view and FIG. 21B is a schematic side view and in both views the schematic representation of the first digital wedge, the second distal wedge, the first proximal wedge and the second proximal wedge of the outer member have been removed for clarity;

FIGS. 22A and 22B are schematic illustrations showing the physical relationship between a first internal member and a second internal member of the outer member of FIG. 14. FIG. 22A is a schematic front view and FIG. 22B is a schematic side view and in both views the schematic representation of the first digital wedge, the second distal wedge, the first proximal wedge and the second proximal wedge of the outer member have been removed for clarity;

FIGS. 23A and 23B are schematic illustrations showing the physical relationship between a first internal member and a second internal member of the outer member of FIG. 14. FIG. 23A is a schematic front view and FIG. 23B is a schematic side view and in both views the schematic representation of the first digital wedge, the second distal wedge, the first proximal wedge and the second proximal wedge of the outer member have been removed for clarity;

FIG. 24 is a schematic illustration of portions of the outer member of FIG. 14, including a first digital wedge, a second distal wedge, a first proximal wedge and a second proximal wedge, along with a first internal member, a second internal member, and an effector tool. This schematic illustration shows movement of the first digital wedge, the second distal wedge, the first proximal wedge and the second proximal wedge after both the first internal member and the second internal member have been activated;

FIGS. 25A and 25B show an exemplary embodiment of components of a fastener for use with an embodiment of a monocortical orthopedic locking system of the present disclosure in a first unexpanded state;

FIGS. 26A-26C show the fastener of FIG. 25A in a second expanded state;

FIGS. 27A and 27B show a close-up view and a cutaway view of the fastener of FIG. 26A;

FIG. 28 is a schematic illustration of a cross-sectional perspective view showing the fastener of FIG. 26A being used as part of a monocortical orthopedic locking system of the present disclosure;

FIGS. 29A and 29B are schematic illustrations of the fastener of FIG. 26A in use with a monocortical orthopedic locking system of the present disclosure showing a locking cap to lock the fastener and a bone plate to one another. Certain features of the monocortical orthopedic locking system are not visible for clarity; and

FIGS. 30A and 30B show an exemplary embodiment of components of a fastener for use with an embodiment of a monocortical orthopedic locking system of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Systems and methods for bone fixation are disclosed herein. In some embodiments, a monocortical orthopedic locking system of the present disclosure is used as an ancillary fracture fixation with a photodynamic bone stabilization system (PBSS). In some embodiments, the monocortical orthopedic locking system can include a plate positioned along a bone having a fracture, wherein the fracture has been repaired using a PBSS. A PBSS is a technique that allows clinicians to repair long bone fractures using a light activated, biocompatible monomer contained within an inflatable balloon catheter. Upon the activation of a visible light source, the monomer infused balloon begins to cure. The cured internal bone fixation implant provides both longitudinal strength and rotational stability to allow for fracture repair and stabilization.

In some embodiments, a cured internal bone fixation implant fills the canal of a bone and then a monocortical orthopedic locking system of the present disclosure is used as an ancillary fracture fixation system. A monocortical orthopedic locking system of the present disclosure can include an elongated bone plate having an upper surface, a lower surface for positioning to face a bone, and a plate hole extending from the upper surface to the lower surface and a fastener that is positioned within a hole of the bone plate and into a cured internal bone fixation implant. In some embodiments, multiple fasteners of the present disclosure are placed in the bone plate holes and grip the cured internal bone fixation implant, thereby causing compression and healing of the weakened or fractured bone. In some embodiments, the grip that the cured internal bone fixation implant provides to the fasteners is greater than that of bone alone, thus there isn't a need for bi-cortical drilling in which both sides (cortices) of the bone are drilled through. The configuration of the photodynamic intramedullary implant can maximize the fastener holding power of the photodynamic intramedullary implant relative to the bone.

In some embodiments, the systems described herein negates the need to measure for each fastener depth. Using the proposed fasteners, a standard depth is drilled such that all of the drill holes shall be the same depth. In some embodiments, a fastener of the present disclosure is placed in each hole drilled into the bone and implant, attached with hardware such as locking buttons, and then the fastener is secure.

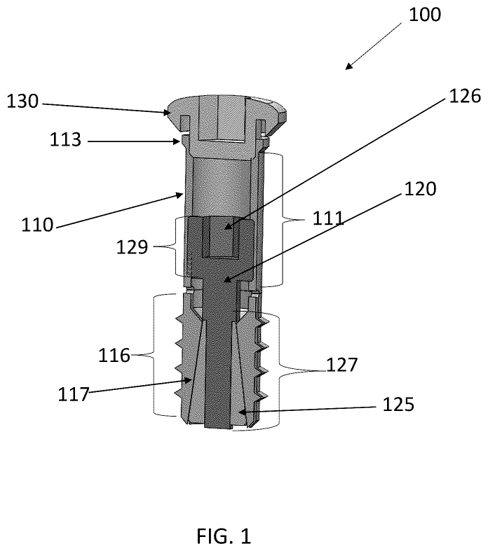

Various types of bone fasteners can be used with a monocortical orthopedic locking system of the present disclosure. FIG. 1 illustrates an exemplary embodiment of components of a bone fastener 100 for use with a cured internal bone fixation implant sufficiently designed to repair a weakened or fractured bone. As shown in the cross-sectional image of FIG. 1, the bone fastener 100 includes an outer member 110 and an internal member 120. The outer member 110 has a first outer section 111 and a second outer section 116, wherein formed in the first outer section 111 is a first bore and formed in the second outer section 116 is a second bore. In an embodiment, the first bore is a simple through hole. In an embodiment, the second bore is a tapered hole.

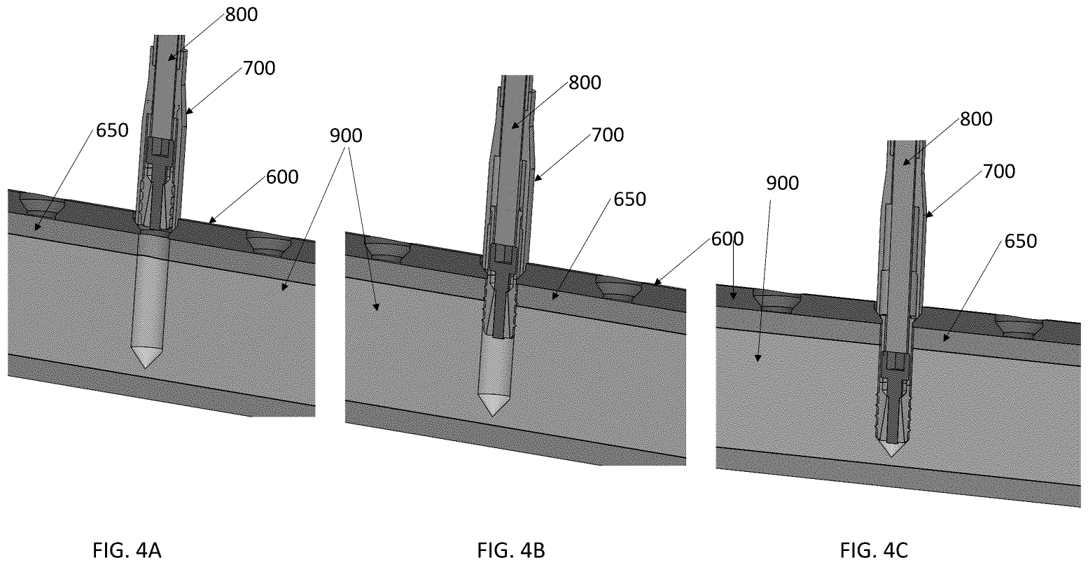

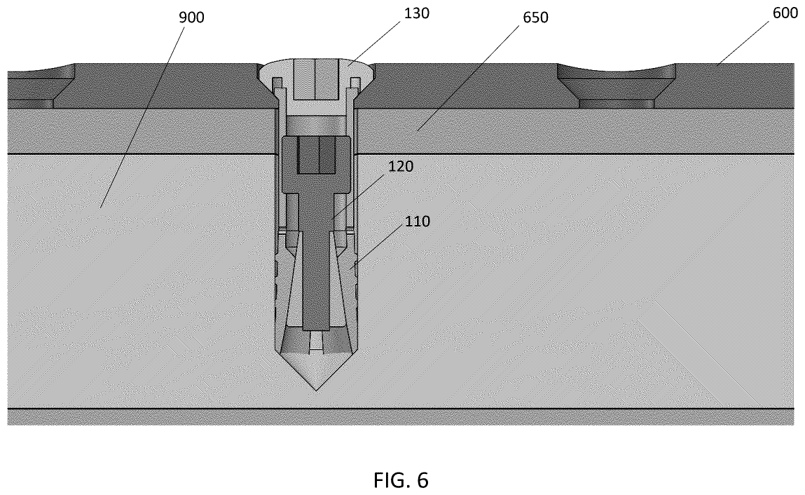

The internal member 120 is a member that is capable of longitudinal movement with respect to the outer member 110 when a pulling force is applied to it. Internal member 120 has a first internal section 127 having an outer surface that is sufficiently designed to fit within and engage a wall of the second bore of the second outer section 116 of outer member 110 and a second internal section 129 sufficiently sized to fit within and engage the first bore of the first outer section 111 of outer member 110. In some embodiments, the first internal section 127 lis a flared distal head. The size and shape of the flared distal head is such that the internal member 120 cannot come loose and cannot be disengaged prior to the activation of the fastener 100. The second internal section 129 of the internal member 120 has an outer end with a peripheral engagement portion 126 being shaped and dimensioned for mating engagement with an effector tool 800, as shown in FIGS. 4A-4C. The second outer section 116 includes an inner tapered profile 117 complimentary to an outer wedge feature 125 of the first internal section 127 of the internal member 120. In order to lock the fastener 100 to a bone plate, a locking button or cap 130 can be positioned on the outer member 110, as shown in position in FIG. 6.

The length of the outer member 110 can vary depending on the depth of the bone and the implant into which the outer member 110 is to be inserted. In some embodiments, the outer member 110 is delivered into a pre-drilled hole in bone. The outer member 110 is configured to move between a first unexpanded position in which the diameter of the outer member 110 is substantially the same along the entire length of the outer member 110 and where the diameter is substantially equal to or slightly smaller than the diameter of the pre-drilled hole, and a second expanded position in the second outer section 116 of the outer member 110 is pushed outward such that it engages with the implant into which the outer member 110 is inserted.

The second outer section 116 of the outer member 110 is compressible from the inside out. In some embodiments, an outer surface of the second outer section 116 of the outer member 110 has surface features thereon that are designed to grip the implant in the bone. In some embodiments, the outer surface of the second outer section 116 of the outer member 110 has a roughened surface. In some embodiments, the second outer section 116 of the outer member 110 may have metallic inserts to enhance the grip forces on the implant. The second outer section 116 of the outer member 110 can include features on the outer surface thereof to enhance the engagement with bone and/or the implant. In some embodiments, the second outer section 116 of the outer member 110 can include ribs/detents on the outer surface so that the compressed surfaces are holding themselves in position once the upwards force has been applied to the outer member 110. The orientation of the ribs/detents on the outer member 110 can vary. The ribs/detents on the outer surface can be applied in a 0-180 degree orientation.

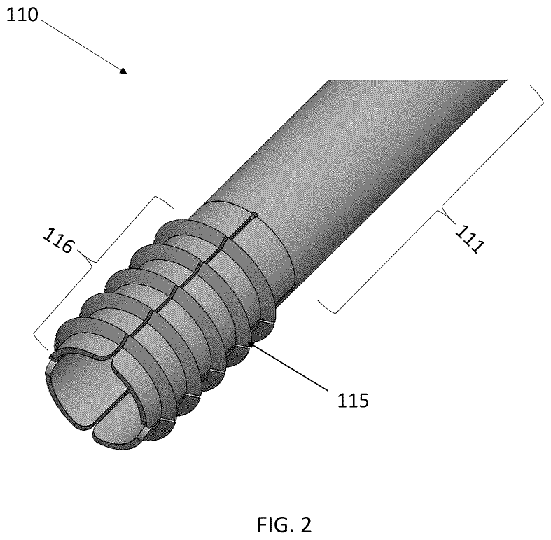

In some embodiments, a plurality of threads 115 are formed on the outside of the second outer section 116 of the outer member 110, as shown in FIG. 2. In some embodiments, the size of the threads 115 can vary along the length of the outer member 110. For example, the threads 115 may be formed so that the most distal ribs are slightly smaller than the proximal ribs such that each thread 115 is compressed into the implant at the same degree. The size difference between the threads 115 to achieve this can be based upon the angle of deformation of the second outer section 116 of the outer member 110 caused by the internal member 120. In some embodiments, the lower or distal ribs may extend slightly further outwards to take advantage of the greater compression at the lower rim to achieve a greater interference fit with the bone.

In some embodiments, the outer member 110 can include a depth stop to ensure that the system is delivered at a level where the top of the outer member 110 is flush with the surface of the bone. In some embodiments, the depth stop can be in the form of a flared proximal end of the outer member 110, such as lip 113 shown in FIG. 1.

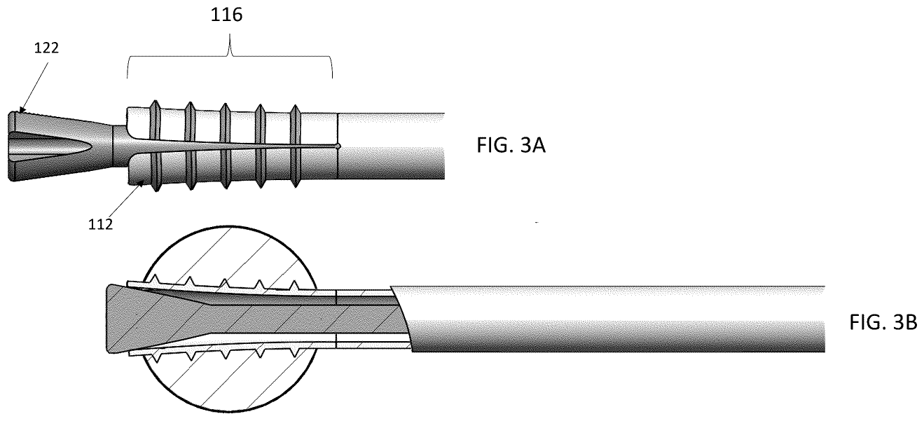

As shown in FIGS. 3A and 3B, in some embodiments, initially the internal member 120 is positioned inside the outer member 110 with distal head 122 extending beyond distal end 112 of the outer member 110. This allows the outer member 110 to remain in its first unexpanded position during insertion of the fastener 100. As the internal member 120 is pulled proximally, as shown in FIG. 3B, the flared shape of the distal head pushes the receiving region 116 of the outer member 110 outward and into its second expanded position.

In some embodiments, the internal member 120 is a metal rod. The metal rod is configured to protrude from the outer member 110. In some embodiments, the internal member 120 can include surface features to enhance the pulling forces of the internal member 120 to expand the outer member 110. For example, the distal portion of internal member 120 can have ribs/fins to engage with a series of detents in the outer member 110, to hold it in a pre-positioned state.

FIGS. 4A-4C illustrate an embodiment of a monocortical orthopedic locking system of the present disclosure. The monocortical orthopedic locking system comprises a bone plate 600 with holes that accept a plurality of the fasteners of FIG. 1 to help stabilize the internal fixation implant 900. The bone plate 600 is positioned on the external surface of a bone 650 that has received a cured internal fixation implant 900. FIGS. 4A-4C show fasteners 100 being positioned with the aid of a guide instrument 700. Also visible is an effector tool 800 for moving (e.g., pushing, pulling, or both pushing and pulling) the internal member 120. In an embodiment, fasteners 100 can be positioned by guide instrument 700 via a trigger. In an embodiment, the guide instrument 700 is configured to hold the effector tool 800, where the outer member 110 is delivered into the pre-drilled hole. The effector tool 800 can be used to apply a force to the internal member 120 of the fastener 100 to engage with and expand the expansion features 116 of the outer member 110 In some embodiments, the guide instrument 700 is configured to mount to the effector tool 800. The guide instrument 700 is configured to removably couple to the effector tool 800 to allow the guide instrument 700 to pull on the internal member 120 and push against the outer member 110. The guide instrument 700 and effector tool 800 can detach from the internal member 120 using a variety of techniques. In some embodiments, the guide instrument 700 and/or the effector tool 800 are detachably coupled to the internal member 120 using a weakened component or connection point, such as a detent, such that enough force on the connection will detach the tools from the internal member 120. In some embodiments, the guide instrument 700 is a separate component that can disengage from the internal member 120. For example, there can be corresponding threads or other features on the guide instrument 700 and the internal member 120 that allow the guide instrument 700 to be removed from the internal member 120 after activation of the outer member 110. The pulling motion on the guide instrument 700 causes the guide instrument 700 to pull the internal member 120 in a proximal direction causing the distal end of the internal member 120 to advance proximally relative to the outer member 110. This causes the distal portion of the outer member 110 to expand outward into the second expanded position to engage with the implant and/or bone.

The ergonomics of the tip of the guide instrument 700 are such that it compensates for non-alignment of the instrument 700 tip to the effector tool 800. For example, most instruments are axial but could be a joint so you can pull on the fastener with the guide instrument 700 with a hinge between then. This configuration of the guide instrument 700 can be used, for example, if the screw hole drilled into the implant/bone is misaligned, allowing for compensation for angulation of the drilled hole and placement of the fastener. For example, a flared configuration allows the guide instrument 700 and effector tool 800 to be off axis.

In some embodiments, a guide instrument of the present disclosure has a cutting tool to remove any extension of the effector tool that is longer than necessary. In some embodiments, the guide instrument design is such that the fasteners are loaded in a bandolier fashion, with the fasteners being loaded for multiple application by the surgeon without having to individually reload the fasteners.

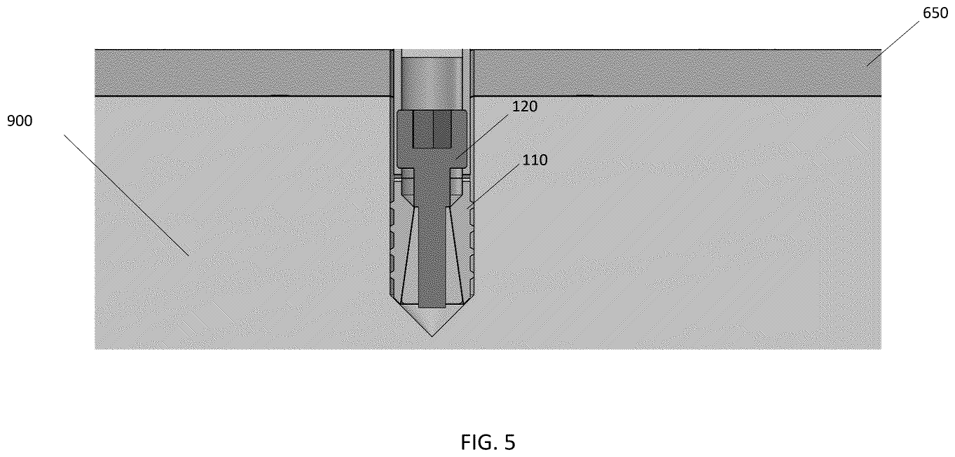

FIG. 5 is a schematic illustration of a close-up cross-sectional side view of the fastener of FIG. 1 positioned within the bone and the cured internal bone fixation implant. A guide instrument or an effector tool is not shown for clarity.

FIG. 6 is a schematic illustration of a close-up cross-sectional view of fastener 100 fully seated within a hole to span the bone 650 and the internal fixation implant 900. In FIG. 6, the outer member 110 is in a second expanded position as the internal member 120 has been pulled in a proximal direction to allow the distal head 122 (first internal section 127) of the internal member 120 to be pulled relative to the outer member 110, causing the distal portion 112 (second outer section 116) of the outer member 110 to flare outward.

In some embodiments, the system further includes a locking mechanism, such as button 130, which can be used to lock a fastener and the bone plate to one another. In some embodiments, the button 130 can be locked using a twisting motion to push the plate downward against the bone surface. In some embodiments, the button 130 can include threads, or can be in form of a bayonet fastener. The locking mechanism 130 is configured to hold some compression as the bone plate 600 can load onto the fasteners 100 and the implant 900 in the bone 650. The length of the locking mechanism 130 can vary, but in some embodiments, the length of the locking mechanism 130 is such that the distal end of the locking mechanism 130 will extend into the fastener 100 a depth at which the implant is positioned in the bone. This allows the locking mechanism to engage the implant.

In some embodiments, the buttons 130 have a shape that conforms to the curvature shape of the fastener. The curved shape makes a button low profile and level with the plate. In some embodiments, the distal portion of the button can be elongated and extended so as to apply counter pressure to the outer member as the button is being driven downwards. The effector tool is pulled upwards, but a counter force is applied by the guide instrument and/or by the button so that the outer member is not lifting upwards as it is being expanded. The button is driven downwards by an action that is pulling the effector tool upwards to drive the button down into position.

In some embodiments, the button can be a wave washer that is advanced by the guide instrument or effector tool, squeezing of the guide instrument or effector tool, is moving the wave washer downwards. A guide instrument or effector tool of the present disclosure can have grips on the circumference to further ensure gripping characteristics of the wave washer. In some embodiments, the button can be threaded so that the button is screwed downwards. In some embodiments, the button has engagement pins for rotation. These can be holes/slots/grooves to accept an instrument that can apply rotation to the button as it is spun into position. The spinning of the button into place can be caused by the guide instrument or effector tool. The buttons are able to tension the lower elements of the fastener in place within the implant filled with a button, locking the system in place. In some embodiments, the engagement points for the guide instrument or effector tool are all low profile so as not to cause tissue damage.

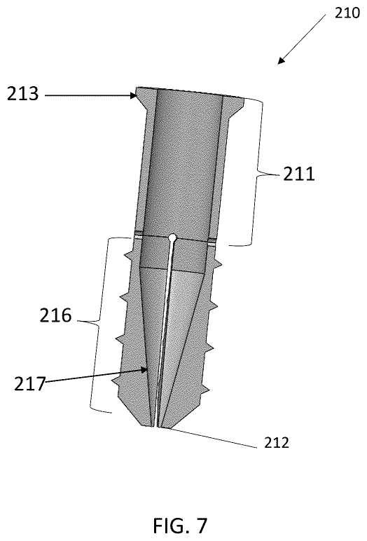

In some embodiments, rather than a pulling motion to activate an outer member of a fastener, a pushing motion can be used. FIG. 7 is a cross-sectional view of an exemplary embodiment of an outer member 210 of a fastener of the present disclosure for use with a monocortical orthopedic locking system. The outer member 210 has a first outer section 211 and a second outer section 216, wherein formed in the first outer section 211 is a first bore and formed in the second outer section 216 is a second bore. In an embodiment, the first bore is a simple through hole. In an embodiment, the second bore is a tapered hole. In some embodiments, the outer member 210 has a depth stop 213 to ensure that the fastener is delivered at a level where the top of the outer member 210 is flush with the surface of the bone.

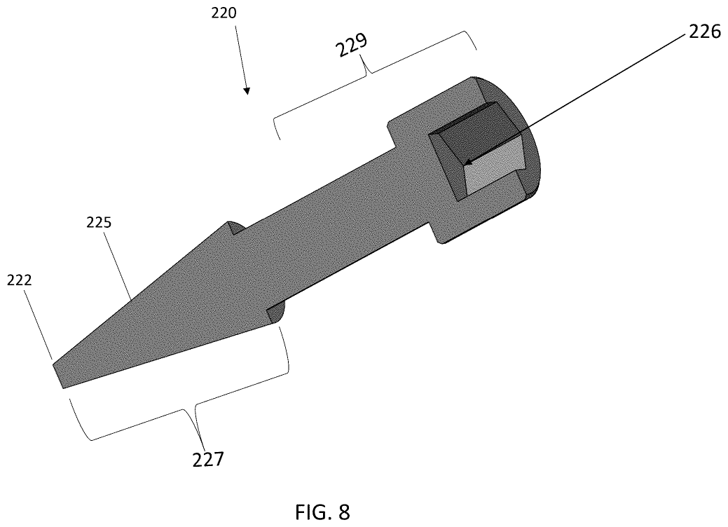

FIG. 8 shows a cross-sectional image of an internal member 220 moveable within the outer member 210. Internal member 220 has a first internal section 227 having an outer surface that is sufficiently designed to fit within and engage a wall of the second bore of the second outer section 216 of outer member 210 and a second internal section 229 sufficiently sized to fit within and engage the first bore of the first outer section 211 of outer member 210. Together outer member 210 and internal member 220 make up components of a fastener 200. In some embodiments, the first internal section 227 is a tapered distal head. The second outer section 216 of outer member 210 includes an inner tapered profile 217 complimentary to an outer wedge feature 225 of the first internal section 227 of the internal member 220. The outer member 210 has a lip 213 at its proximal tip to prevent the outer member 210 from being inserted too deeply into a bone/internal bone fixation system.

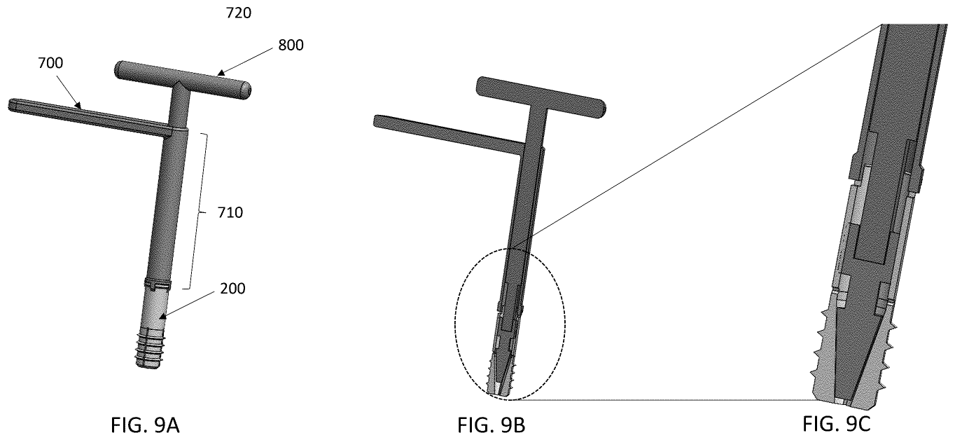

As shown in FIGS. 9A-9C, a guide instrument 700 can include a stabilizing portion 710 that is configured to engage with the proximal end of a fastener of the present disclosure to hold the fastener in place in the pre-drilled hole during the application of force by the guide instrument 700. A handle is formed on the proximal end of the effector tool 800 and is used to apply the force to an internal member of the present disclosure. The effector tool 800 releasably engages an internal member of the present disclosure and can transfer either a pulling force, a pushing force, or both depending on the embodiment of fastener being used.

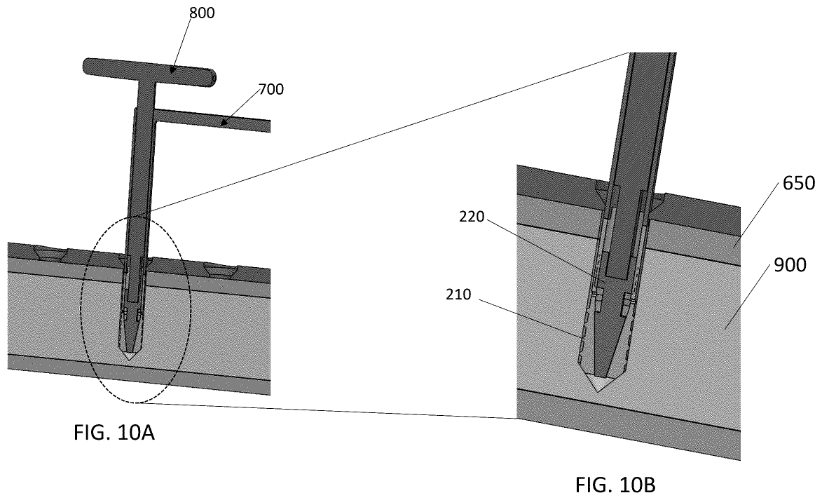

FIGS. 10A and 10B illustrate cross-sectional perspective views of an exemplary embodiment of the use of the fastener of FIG. 9B as a component of a monocortical orthopedic locking system to fix an internal bone fixation implant 900 with bone 650.

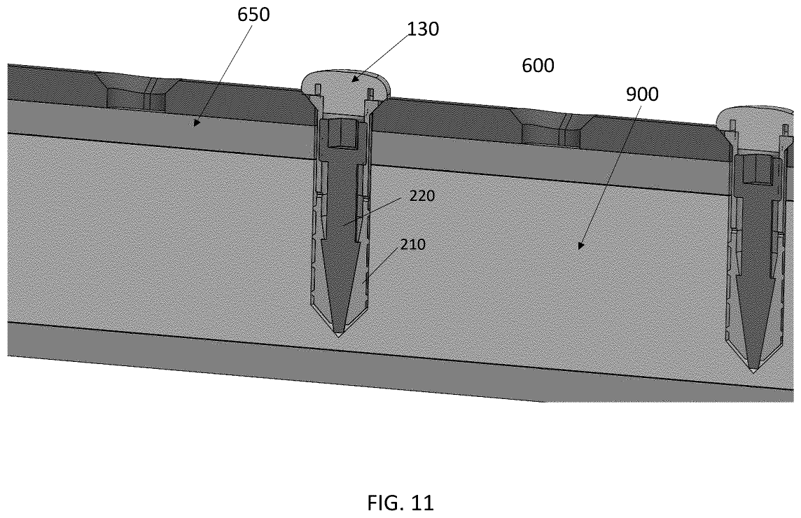

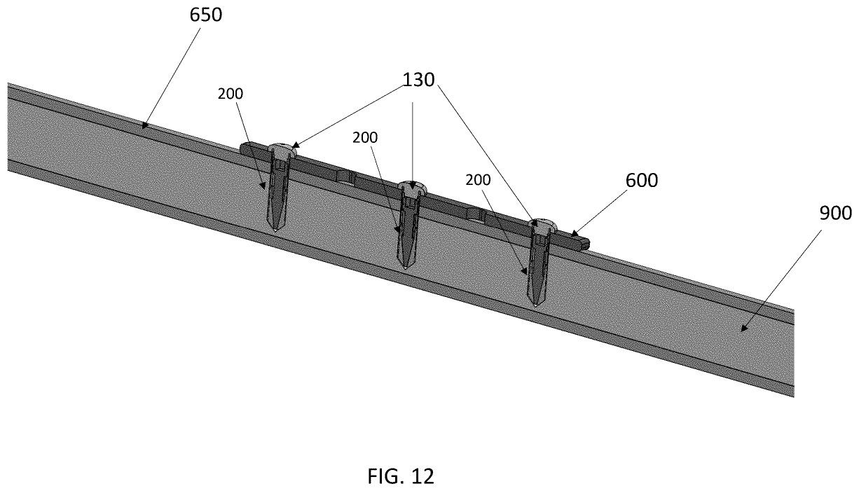

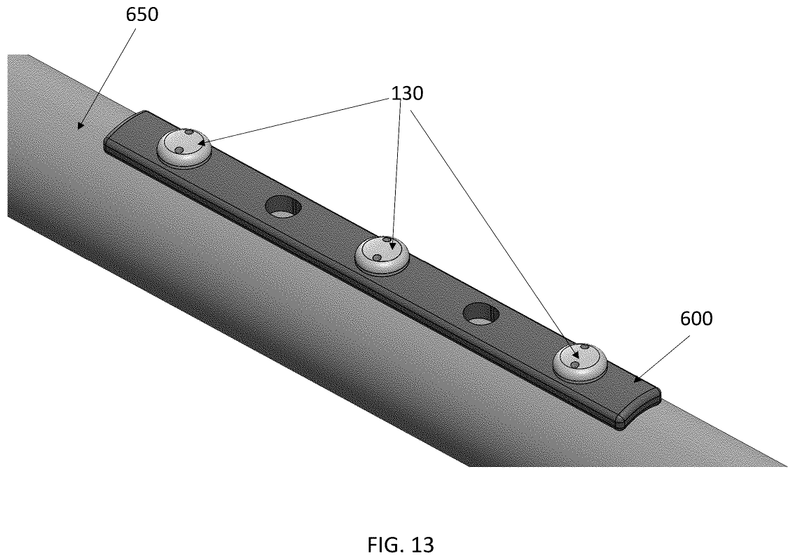

FIGS. 11-13 illustrate the use of fastener 200 with an internal fixation implant positioned in a bone. FIG. 11 illustrates the outer member 210 in the second expanded position as the internal member 220 has been pushed in a distal direction to allow the distal head 222 of the internal member 220 to be pushed past the distal end 212 of the outer member 210, causing the second outer section 216 of the outer member 210 to flare outward. The internal member 220 is configured to be positioned in the outer member 210 such that the wedge feature 225 is pushed into the complimentary inner tapered profile 217 of the expansion tube 210 and to cause the outward expansion of the distal end of the outer member 210. FIGS. 12 and 13 illustrate a plurality of fasteners 200 positioned in bone and coupled to a fixation implant to further stabilize the bone along a length thereof.

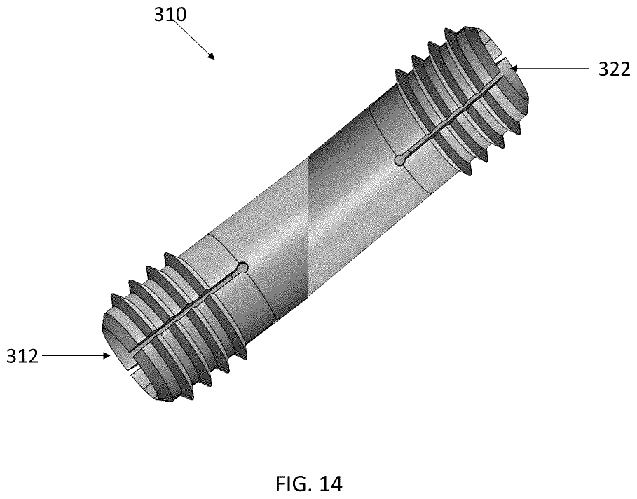

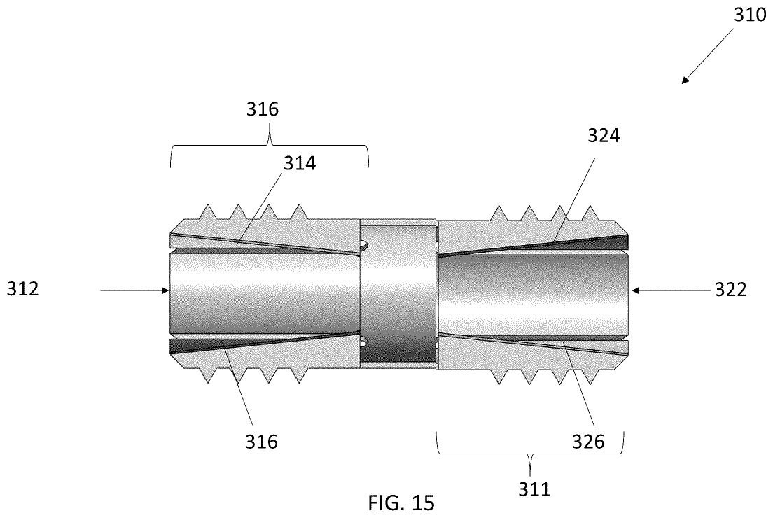

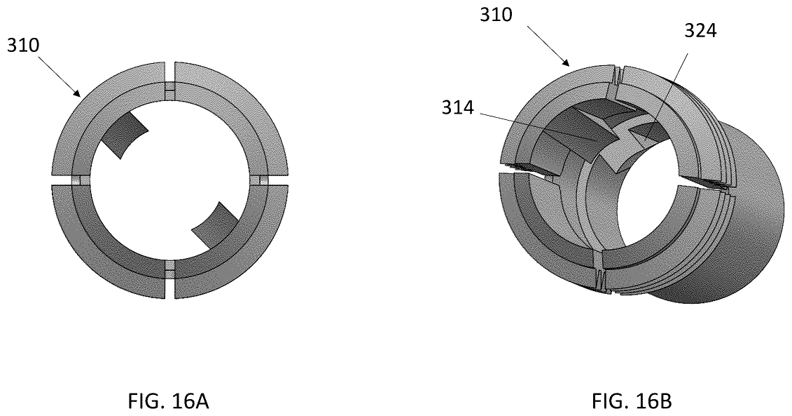

In some embodiments, a monocortical orthopedic locking system of the present disclosure uses a fastener having an outer member that utilizes both a push and pull motion for placement, such as outer member 310 shown in FIGS. 14, 15, 16A, and 16B. FIG. 14 illustrates outer member 310 having a distal end 312 and a proximal end 322. FIG. 15 illustrates a cross-sectional view of the push-pull outer member 310. The outer member 310 includes a first outer section 311 and a second outer section 316, wherein formed in the first outer section 311 is a first bore and formed in the second outer section 316 is a second bore. In an embodiment, the first bore and the second bore have the same size and dimension. The outer member 310 has a first proximal wedge 324 and a second proximal wedge 326 positioned in the first outer section 311 and a first distal wedge 314 and a second distal wedge 315 positioned in the second outer section 316.

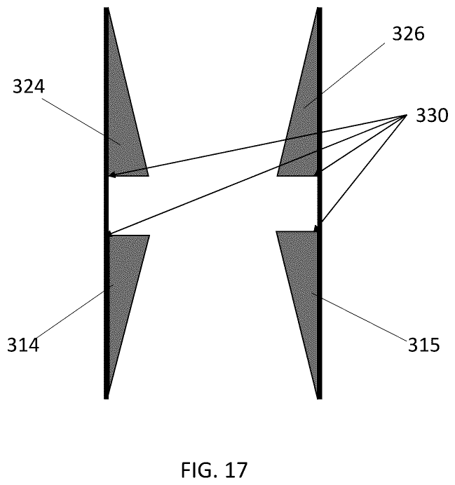

The length of the outer member 310 can vary depending on the depth of the bone into which the outer member 310 is to be inserted. The outer member 310 is delivered into a pre-drilled hole in bone. FIG. 17. 18 and 19 are schematic illustrations of an exemplary embodiment of the expandable portions of the outer member 310. The outer member 310 includes the first and second distal wedges 314 and 315 in line with the first and second proximal wedges 324 and 326 on an inner surface of the outer member 310. The proximal ends of the first and second distal wedges 314 and 315 and the distal ends of the first and second proximal wedges 324 and 326 include deformation points 330 such that the wedges expand outward at the deformation points to engage an internal bone fixation implant and/or cortical bone.

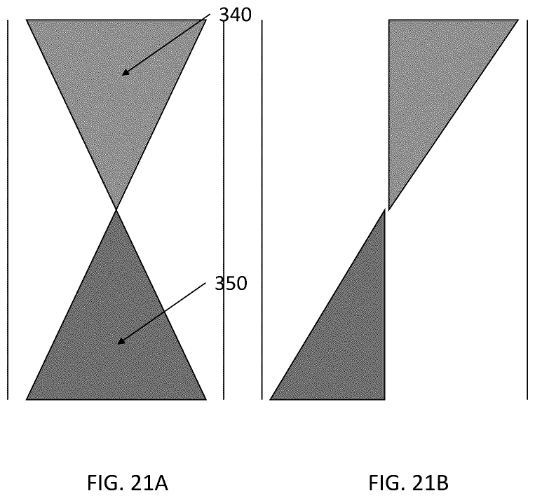

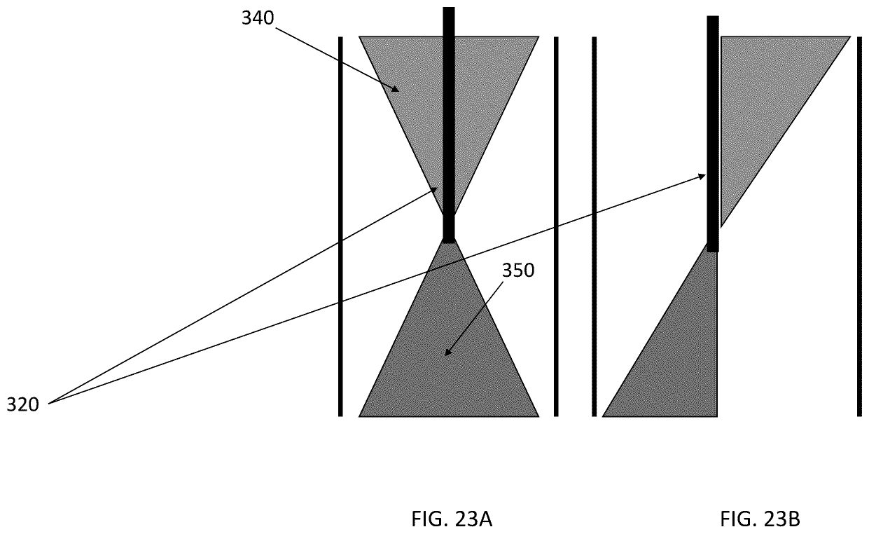

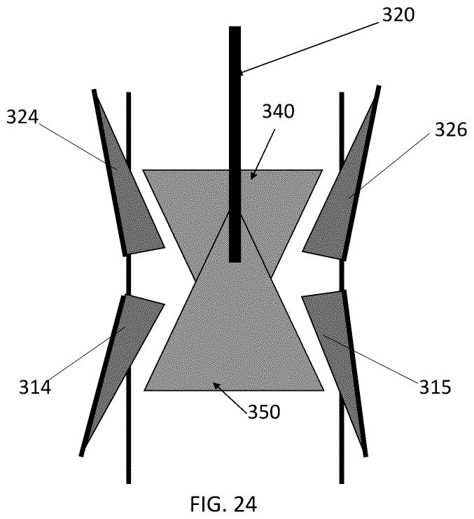

The outer member 310 is configured to move between a first unexpanded position, shown in FIGS. 17-23B, in which the outer diameter of the outer member 310 is substantially the same along the entire length, and a second expanded position, schematically illustrated in FIG. 24, in which the internal wedges 314, 315, 324, and 326 of the outer member 310 are expanded outward via internal movement of the proximal expander 340 and distal expander 350 such that the first outer section 346 and the second outer section 336 are flared out to engage with the bone and the internal bone fixation implant.

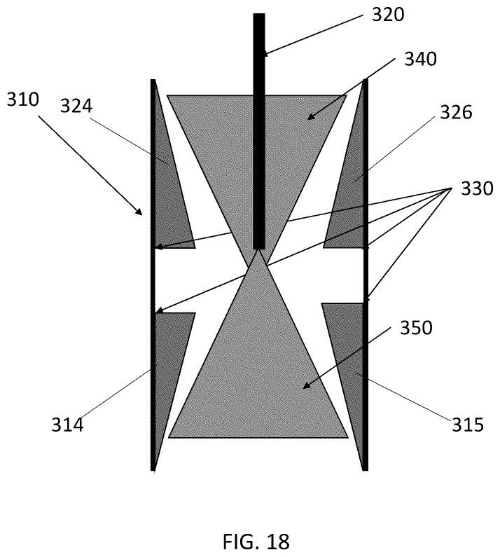

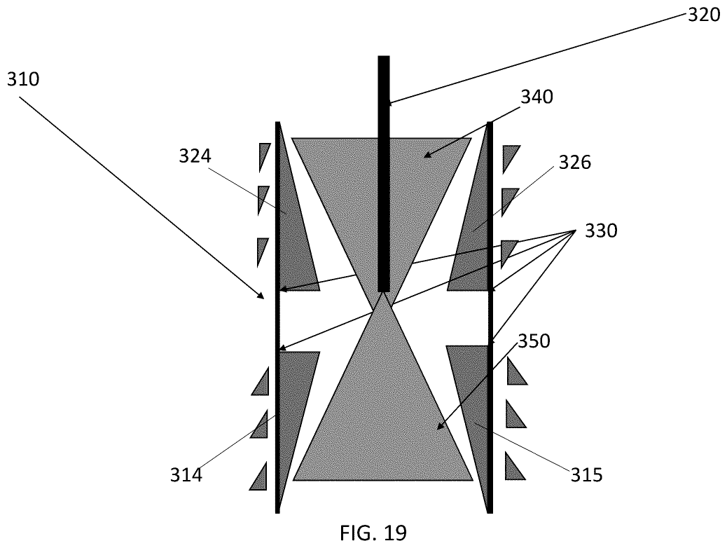

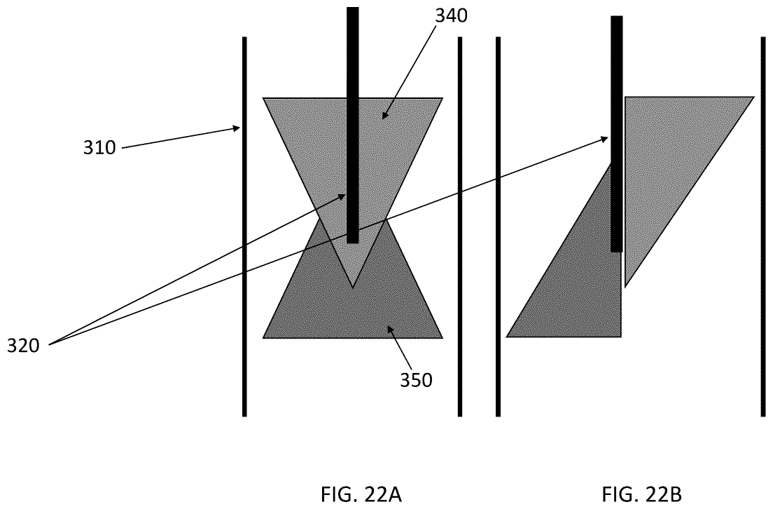

To move the outer member 310 into the second expanded position, an internal member 320 is used. As shown schematically in FIGS. 18 and 19, the internal member 320 can include a proximal expander 340 (expander wedge, or triangular form) and a distal expander 350 (expander wedge, or triangular form). The proximal expander 340 is configured to move distally to activate the first and second proximal wedges 324 and 326 of the outer member 310, and the distal expander 350 is configured to move proximally to activate the first and second distal wedges of the outer member 310. In some embodiments, the proximal expander 340 and the distal expander 350 are positioned such that they pass each other and engage the expansion tube from the distal and proximal portions, as shown in FIGS. 22A and 22B. The motion of the proximal expander 340 and the distal expander 350 is driven by an effector tool, which can pull on the distal expander 350 while pushing on the proximal expander 340. The distal form of the expander couples to a portion of the applier, such as a distal effector tool that extends from it which is used by the applier to pull the distal form upwards. The proximal form of the expander couples to or engages with a portion of the applier, such as a proximal effector tool that extends from it which is used by the applier to push the proximal form downwards.

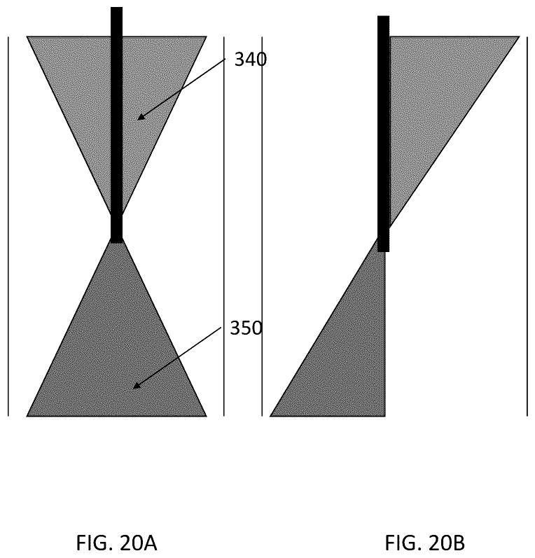

For example, as shown in FIGS. 20A, 20B, 21A, 21B, 22A, 22B, 23A and 23B, slidable triangular forms 340 and 350 are configured to pass each other, engaging the outer member 310 from the top and the bottom. The motion of the two forms 340 and 350 is driven by the effector tool. In some embodiments, the effector tool is pulling on the bottom form 350 and pushing on the top form 340. The lower form 350 can have 320 extending from it, which is used to pull the lower form 350 upwards, where a locking button can then be driven down onto it affixing the system in place.

FIG. 24 illustrates an exemplary embodiment of the fastener 300 shown in the second expanded configuration after the instrument/tool has moved both the proximal and distal expanders 340 and 350 simultaneously to lock the fastener 300 in place relative to the implant/bone. After the outer member 31 is in the expanded position, a button can be driven down onto the fastener, affixing the fastener and the implant in place.

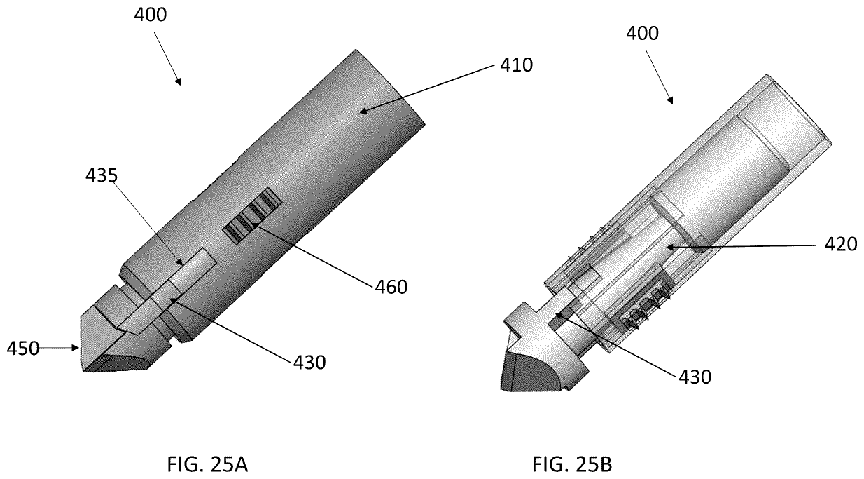

FIGS. 25A and 25B show an embodiment of some of the components of a fastener 400 of the present disclosure. Fastener 400 includes an outer member 410 and an internal member 420. The distal end of internal member 420 is in the form of a self-drilling tip 450 that has a side cutting edge 430. The proximal end of internal member 420 can be a drive shaft. The outer member 410 includes slot 435 at a distal end thereof that allows the cutting edge 430 of the internal member 420 to ride within, other additional slots and cutting edges can be used. The cutting edges 430 are slightly larger than the circumference of the outer member 410, and provides enough span for the outer member 410 to be inserted into bone.

Self-drilling fastener 400 allows for the simultaneous drilling of holes in bone and into a cured internal bone fixation implant with insertion of the fastener. Outer member 410 includes one or more components 460 configured to engage with bone and a cured internal bone fixation implant.

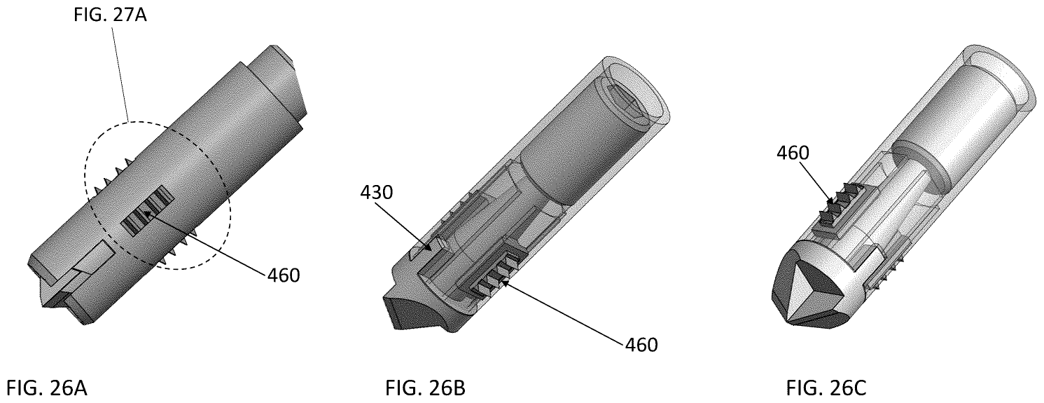

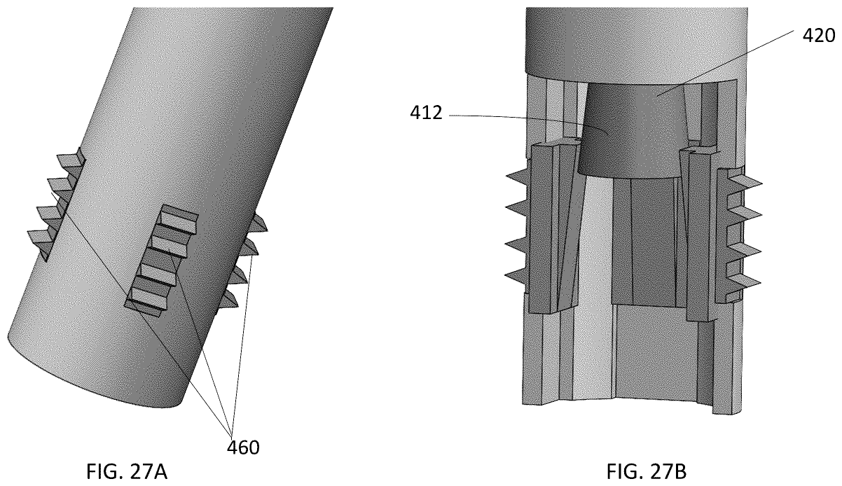

In some embodiments, component 460 consists of a piece of material that has a back face with toothed wings and a front face with an inclined ramp shape. Each component is designed such that the back face with the toothed wings extend through openings in the walls of the outer member 410 and the front face with an inclined ramp shape is positioned in the internal hollow of the outer member 410, as shown in FIG. 27B. The internal member 420 is shaped and dimensioned to fit within the internal hollow of the outer member 410 and will be positioned in the internal hollow such that the distal end 412 is initially at a location that is distal relative to the components 460, similar to the starting position seen in the fastener shown in FIG. 3A. A distal end 412 of the internal member 420 has a flared shape such that pulling the internal member 420 up causes the inclined ramp shape of acomponent 460 to move the toothed wings from a first unengaged position (see in FIG. 25B) inside the outer member 410 to a second engaged positioned (FIG. 26B) where the wings extend outside the outer member 410, as shown in FIGS. 27A and 27B. After the fastener 400 is positioned, the drill tip 450 at the distal end 412 of the internal member 420 is retracted into the outer member 410, as schematically illustrated in FIGS. 26A-26C.

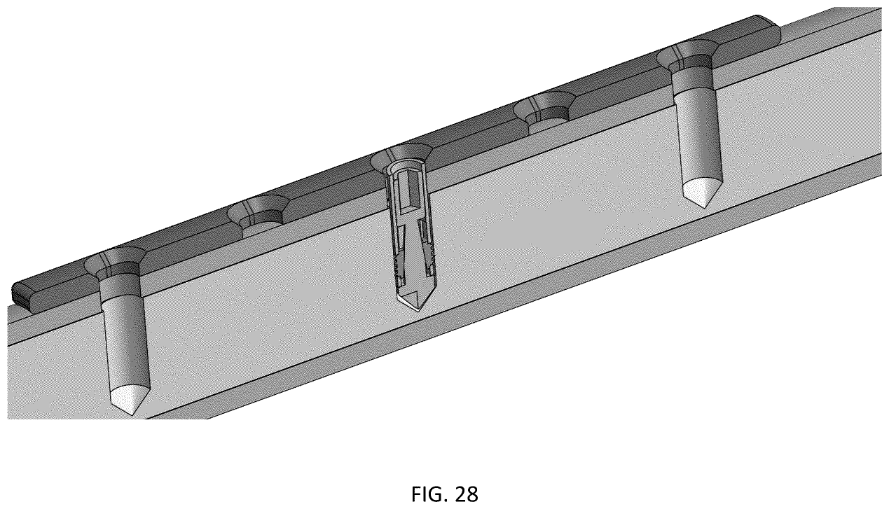

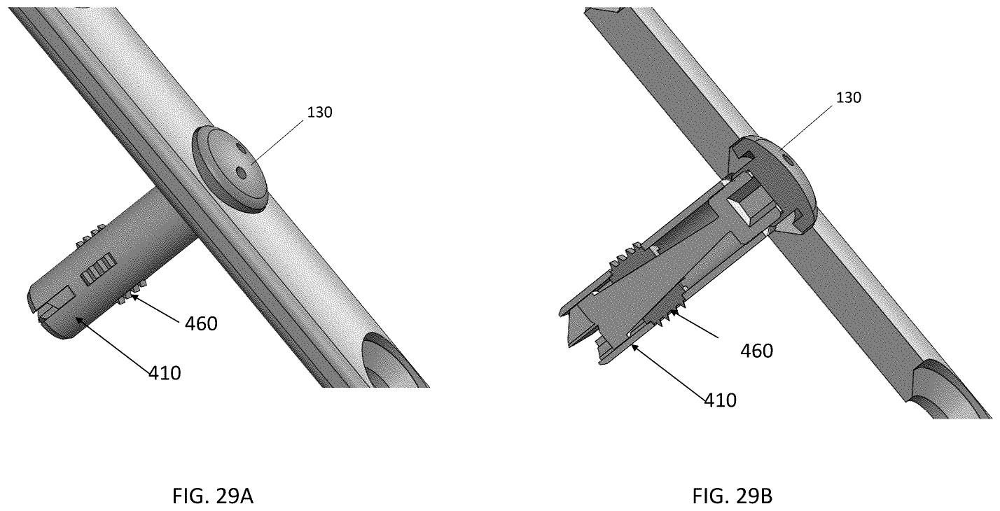

FIG. 28 is a schematic illustration of a cross-sectional perspective view showing the fastener of FIG. 25A being used as part of a monocortical orthopedic locking system of the present disclosure. FIG. 29A (perspective view) and 29B (perspective cross-sectional view) are schematic illustrations of the expanded fastener of FIG. 26A in use with a monocortical orthopedic locking system of the present disclosure including button 130 to lock the fastener and a bone plate to one another. In some embodiments, the button can be locked using a twisting motion to push the plate downward against the bone surface. In some embodiments, the button can include threads, or can be in form of a bayonet connection with the lip of the outer member. The locking mechanism is configured to hold some compression as the bone plate can load onto the fasteners and the implant in the bone. The length of the locking mechanism can vary, but in some embodiments, the length of the locking mechanism is such that the distal end of the locking mechanism will extend into the fastener a depth at which the implant is positioned in the bone. This allows the locking mechanism to engage the implant.

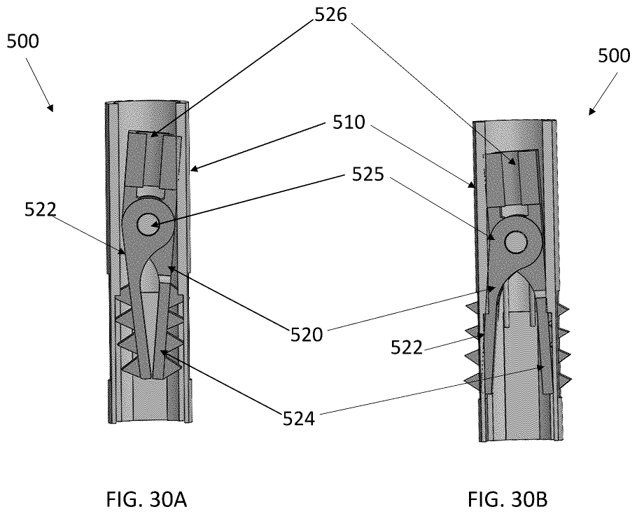

In some embodiments, as shown in FIGS. 30A and 30B, a fastener 500 of the present disclosure includes an outer member 510 and an internal member 520. Internal member 520 has first and second arms (522, 524) coupled at a pivot point 525. Internal member 520 has a peripheral engagement portion 526 being shaped and dimensioned for mating engagement with an effector tool. In use, when an effector tool is longitudinally moved within the outer member 520, it causes movement of the first and second arms 522, 524 from a first unengaged position inside the outer member 510 to a second engaged positioned where the first and second arms 522, 524 pivot relative to one another to allow distal ends of the first and second arms 522, 524 to extend outside the outer member 510 and engage with an internal bone fixation implant and bone. In some embodiments, the distal ends of the first and second arms 522, 524 can include surface features, such as teeth, to engage with the internal bone fixation implant and bone.

A method of using a bone fixation system of the present disclosure includes implanting a deflated conformable member of an internal bone fixation system within a weakened or fractured bone, inflating the member to a desired state with a reinforcing material, and curing the reinforcing material within the member to harden the material and create a hardened implant. The cured hardened implant and bone are then fitted for a bone plate of a bone plating system of the present disclosure, and the bone and implant are drilled for the fastener locations along the length of the plate. The fasteners of a bone plating system of the present disclosure are sufficiently designed as monocortical fasteners that anchor into only one layer of bone cortex, thus the holes are only drilled to a specified depth. For example, the holes are drilled to a depth of at least half the implant diameter to ensure that an expansion tube of a fastener of the present disclosure is placed within sufficient implant material thickness to achieve fixation. The fasteners are delivered through the holes in the plate. The outer member of a fastener of the present disclosure can have a lip to prevent the fastener from being inserted too deeply.

A method for treating a bone includes inserting an inflatable balloon catheter into a medullary canal of the bone; expanding the balloon catheter with a biocompatible monomer; activating a visible light source to cure the monomer and form a cured internal bone fixation implant; placing a bone plate alongside the bone, the bone plate having an upper surface, a lower surface for positioning to face the bone, and a plate first hole extending from the upper surface to the lower surface; inserting a fastener through the first hole, into the underlying bone, and at least into the implant, wherein the fastener comprises: an outer member having a first outer section and a second outer section, wherein formed in the first outer section is a first bore with a first shape and formed in the second outer section is a second bore with a second shape; and an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion; engaging an effector tool with the peripheral engagement portion of the internal member of the fastener; applying a force to the effector tool to longitudinally move the internal member with respect to the outer member to cause the second outer section of the outer member to expand, wherein expansion of the second outer section of the outer member of the fastener results in fixation of the fastener to the implant.

In an embodiment, a fastener of the present disclosure can be removed using an effector tool. The effector tool is moved into position to engage with the peripheral engagement portion of the internal member of the fastener and the internal member can be rotated (e.g., 90 degrees). The rotation of the inner member via rotation of the effector tool 90 degrees can release the outer member so it goes back to an initial unexpanded state.

It is also within the concept of the present disclosure to provide a kit, which includes at least one of the plates and fasteners disclosed herein. The kit can also include additional orthopedic devices and instruments; such as for example, instruments for tightening or loosening the fasteners, including, but not limited to, a guide instrument, an effector tool, locking buttons, and any additional instruments or tools associated therewith. Such a kit can be provided with sterile packaging to facilitate opening and immediate use in an operating room.

All patents, patent applications, and published references cited herein are hereby incorporated by reference in their entirety. It will be appreciated that several of the above-disclosed and other features and functions, or alternatives thereof, may be desirably combined into many other different systems or application. Various presently unforeseen or unanticipated alternatives, modifications, variations, or improvements therein may be subsequently made by those skilled in the art.

What is claimed is:

1. A fastener for use in securing a bone plate, the fastener comprising:
an outer member having a first outer section and a second outer section, wherein formed in the first outer section is a first bore with a first shape and formed in the second outer section is a second bore with a second shape; and
an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion.

2. The fastener of claim 1, wherein an outer surface of the second outer section of the outer member is threaded.

3. The fastener of claim 1, wherein the first shape of the first bore is different from the second shape of the second bore.

4. The fastener of claim 1, wherein the first shape of the first bore is same as the second shape of the second bore.

5. The fastener of claim 1, wherein the internal member has a first internal section that is flared.

6. The fastener of claim 1, wherein the internal member has a first internal section that is tapered.

7. The fastener of claim 1, wherein the peripheral engagement portion of the internal member is shaped and dimensioned for mating engagement with an effector tool.

8. The fastener of claim 1, wherein the outer member is sufficiently designed to releasably engage with a guide instrument.

9. A bone fixation system comprising:
an elongated plate having an upper surface, a lower surface for positioning to face a bone, and a plate bore hole extending from the upper surface to the lower surface; and
a first fastener sized to be inserted through the plate bore hole and having:
an outer member having a first outer section and a second outer section, wherein formed in the first outer section is a first bore with a first shape and formed in the second outer section is a second bore with a second shape; and
an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion.

10. The bone fixation system of claim 9, further comprising a locking cap for positioning at a proximal end of the fastener.

11. The bone fixation system of claim 9, wherein an outer surface of the second outer section of the outer member of the fastener is threaded.

12. The bone fixation system of claim 9, wherein the first shape of the first bore of the outer member of the fastener is different from the second shape of the second bore of the outer member of the fastener.

13. The bone fixation system of claim 9, wherein the internal member of the fastener has a first internal section that is flared.

14. The bone fixation system of claim 9, wherein the internal member of the fastener has a first internal section that is tapered.

15. The bone fixation system of claim 9, wherein the peripheral engagement portion of the internal member is shaped and dimensioned for mating engagement with an effector tool.

16. The bone fixation system of claim 9, further comprising a guide instrument.

17. A method for treating a bone, comprising:
inserting an inflatable balloon catheter into a medullary canal of the bone;
expanding the balloon catheter with a biocompatible monomer;
activating a visible light source to cure the monomer and form a cured internal bone fixation implant;
placing a bone plate alongside the bone, the bone plate having an upper surface, a lower surface for positioning to face the bone, and a plate first hole extending from the upper surface to the lower surface;
inserting a fastener through the first hole, into the underlying bone, and at least into the implant, wherein the fastener comprises:
an outer member having a first outer section and a second outer section, wherein formed in the first outer section is a first bore with a first shape and formed in the second outer section is a second bore with a second shape; and
an internal member longitudinally moveable within the outer member, the internal member having a first internal section having an outer surface that is sufficiently designed to fit within and engage a wall of the second bore of the outer member and a second internal section sufficiently sized to fit within the first bore of the outer member, the second internal section having an outer end with a peripheral engagement portion;
engaging an effector tool with the peripheral engagement portion of the internal member of the fastener;
applying a force to the effector tool to longitudinally move the internal member with respect to the outer member to cause the second outer section of the outer member to expand,
wherein expansion of the second outer section of the outer member of the fastener results in fixation of the fastener to the implant.

18. The method of claim 17, wherein the inserting step further comprises coupling the fastener to an alignment guide instrument.

19. The method of claim 17, further comprising disengaging the effector tool from the peripheral engagement portion of the internal member of the fastener.

20. The method of claim 19, further comprising advancing a locking member until a head of the locking member is fully seated in the first plate hole.
